(19) United States
(12) Patent Application Publication
(10) Pub. No.: US 2024/0252803 A1
(43) Pub. Date: Aug. 1, 2024

(54) CONNECTION WELDING FOR AUTOMATED STERILE CONNECTION AND FLUID TRANSFER

(71) Applicant: Geoffrey L. Hodge, Sutton, MA (US)

(72) Inventor: Geoffrey L. Hodge, Sutton, MA (US)

(21) Appl. No.: 18/566,614

(22) PCT Filed: Jun. 1, 2022
(86) PCT No.: PCT/US22/31764

§ 371 Date: Dec. 1, 2023

Related U.S. Application Data
(60) Provisional application No. 63/196,029, filed on Jun. 2, 2021.

(30) Foreign Application Priority Data
Aug. 6, 2021 (EP) 21193324.7

(51) Int. Cl. A61M 39/14; B29C 65/00

(52) U.S. Cl. CPC A61M 39/146; B29C 66/0018; B29C 66/857

(57) ABSTRACT

Methods and devices for automated sterile connection and liquid transfer between containers. The methods and devices involve welding (e.g., nest welding) to form sterile fluidical connection between the containers. The method includes: (a) placing a first tube and a second tube into a welding mount, wherein the first tube is connected to a first container and the second tube is connected to a second container, and (b) welding the first tube and the second tube to form a first sterile fluidical connection between the first container and the second container. The method also includes (c) transferring a liquid between the first container and the second container via the first sterile fluidical connection. (d) scaling and cutting the first fluidical connection between the first container and the second container to disconnect the first sterile fluidical connection. The devices executing the method can be automated by a controller.

### CROSS-REFERENCE TO RELATED APPLICATIONS

This application claims the benefit of, and priority to, U.S. Provisional Application No. 63/196,029, titled “Connection Welding For Automated Sterile Connection And Fluid Transfer”, filed on Jun. 2, 2021, and European Patent Application No. EP21193324.7, titled “Connection Welding For Automated Sterile Connection And Fluid Transfer”, filed on Aug. 26, 2021, the disclosures of which are incorporated by reference herein in their entirety.

### FIELD

This application relates to a connection welding for automated sterile connection and fluid transfer, e.g., during manufacturing of cells.

### BACKGROUND

Adoptive cell therapy, e.g., chimeric antigen receptor (CAR)-T cell-based therapy, is becoming a promising option for treating various types of cancer because of its potential to evade genetic and cellular mechanisms of drug resistance, and to target tumor cells while sparing normal tissues. Clinical manufacturing of high-quality therapeutic cells is a prerequisite for the wide application of this technology.

Current approaches for producing therapeutic cells for use in adoptive cell therapy typically involve ex vivo enrichment, activation, and expansion of T cells and genetic modification of the T cells using retroviral or lentiviral vectors to introduce an exogenous nucleic acid coding for a chimeric receptor. Sterile connection of thermoplastic tubing by the annealing process of tube welding, e.g. for use in large-scale biotherapeutics manufacturing, has been beneficial to allow subsequent sterile transfer of liquids, however the process of creating a single weld is highly manual and time consuming. Subsequent steps of liquid transfer, sealing, and separation of fluidical flow paths are likewise performed independently of each other as highly manual and time-consuming operations. It is of great interest to develop devices and methods that enable efficient, high-throughput, and cost-effective production of therapeutic cells, e.g. autologous or allogeneic cell therapies.

### SUMMARY

Provided herein are connection welds for sterile automated connection and liquid transfer and methods of use thereof. Connection welds provided herein may be used for sterile liquid transfer during manufacturing of cells to reduce cross-contamination and increase manufacturing productivity and efficiency.

In a first embodiment, a method for sterile automated connection and liquid transfer between two containers includes: (a) placing a first tube and a second tube into a welding mount, wherein the first tube is connected to a first container and the second tube is connected to a second container, and (b) welding the first tube and the second tube to form a first sterile fluidical connection between the first container and the second container. The method also includes (c) transferring a liquid between the first container and the second container via the first sterile fluidical connection, (d) sealing and cutting the first fluidical connection between the first container and the second container to disconnect the first sterile fluidical connection, wherein steps (a)-(d) are operated automatically by a controller. Steps (a)-(d) may be repeated, e.g., operated automatically by the controller.

In a second embodiment, a method for sterile automated connection and liquid transfer between containers, includes: (a) placing a first tube and a second tube into a welding mount, wherein the first tube is connected to a first container and the second tube is connected to a second container, (b) welding the first tube and the second tube to form a first sterile fluidical connection between the first container and the second container, and (c) transferring a liquid between the first container and the second container via the first sterile fluidical connection. The method also includes (d) sealing the first fluidical connection between the first container and the second container and cutting the seal to disconnect the first sterile fluidical connection. Steps (a)-(d) can be repeated for multiple cycles, for example, operated automatically by a controller as disclosed herein. Steps (b) and (d) may include welding, on one side of an existing weld, a selected length of a tubing, and adding new welds to the added tubing until the selected length of tubing is used up. Steps (b) and (d) comprise welding, on one side of an existing weld, a spool piece of a selected length, and adding new welds to the added tubing thereby shortening the length of the spool piece. In an embodiment, wherein steps (b) and (d) comprise welding, on one side of an existing weld, a selected length of a tubing, and adding new welds to the added tubing until the selected length of tubing is used up.

This “nested welding” process can be repeated multiple times to increase the number of welds possible within a given length of tubing.

In a third embodiment, a device includes a welding mount configured to receive a first tube fluidically coupled to a first container and a second tube fluidically coupled to a second container, a welding element configured to fluidically couple the first tube with the second tube by welding, and a pump configured to transfer a liquid from the first container to the second container and a controller comprising a memory circuit storing instructions and a processor circuit configured to execute the instructions.

Generally, the controller comprises a memory circuit storing instructions and a processor circuit configured to execute the instructions and/or a memory circuit storing data of the manufacturing operations and sampling. In some embodiments, the controller schedules the movements based on calculation of analytical in-process data, for example, cell count, cell viability, level of transduction (e.g., via flow cytometry and/or PCR), growth medium properties (e.g., pH, osmolality, and/or metabolites), contaminants (e.g., BSA, DNA, etc. which may be determined during wash steps), time or a combination thereof. In some embodiments, the controller is operated under a scheduling software. For example, the scheduling software is designed to manage dozens to hundreds of cell cultures at the same time. In other examples, the scheduling software is designed to create a custom schedule for the cell culture in each of the cell culture vessels to optimize process performance. In some instances, the optimization of process performance is based on pre-programmed instructions, in-process data, scheduling of sequential use of the workstations, or a combination thereof.

Upon executing the instructions, the controller causes the welding element to fluidically couple the first tube and the second tube, optionally via a fresh “spool piece” of tubing of a selected length. The device further comprises a welding mount configured to receive an automatically loaded piece of fresh tubing/spool piece of a selected length and a single or plurality of welding elements configured to fluidically couple the first tube with one side of the piece of fresh tubing and the other side of the piece of fresh tubing with the second tube by welding wherein all of the steps may be automatically configured by the controller The device also optionally includes an interlock valve configured to avoid back contamination between the first container and the second container, optionally includes a sealing head and cutter to seal and cut the flow path after liquid transfer. The device is optionally configured for use in bioprocessing of cells.

### BRIEF DESCRIPTION OF THE DRAWINGS

Various aspects and embodiments will be described with reference to the following figures. The figures are not necessarily drawn to scale.

FIGS. 1A-1E include schematic depictions of tube welding with source container (100) with associated source container tubing (110), destination container (200) with associated destination container tubing (210) loaded into the weld mounts (300), secured and welded by the Welding blade (400). FIG. 1A depicts the initial positioning, FIG. 1B the cutting by the heated welder blade 400, FIG. 1C the alignment of cut ends after removal of the welder blade, FIG. 1D the welded tubing after withdrawal of the heated welder blade, and FIG. 1E is a photography of a welded tube showing the effect of mushrooming.

FIGS. 2A-2D include schematic depictions of tube welding with auto-loading weld mounts configured for an intermediate/spool piece (500). Weld mounts (310) hold source container tubing (110) with destination container tubing (210) via spool piece/intermediate portion of tubing (500) FIG. 2A depicts the initial positioning, FIG. 2B the cutting by the heated welder blade 400, FIG. 2C the alignment of cut ends after removal of the welder blade, and FIG. 2D the welded tubing after withdrawal of the heated welder blade.

FIGS. 3A-3D include schematic depictions of tube welding with auto-loading weld mounts and a bent intermediate/spool piece (500) using a single welding blade (400). FIG. 3A depicts the initial positioning, FIG. 3B the cutting by the heated welder blade 400, FIG. 3C the alignment of cut ends after removal of the welder blade, and FIG. 3D the welded tubing after withdrawal of the heated welder blade.

FIG. 4 is a schematic depiction of the positioning of a pump (600) and pinch valves (700) for tube welding between contains 100 and 200.

FIG. 5 is a schematic depiction of the tube used for each weld in dependence of the seal width, the gaps and the width of the gripper of the weld mounts.

FIG. 6 is a schematic depiction of liquid transfer of media from left to right to a cell culture container with subsequent sealing, cutting and removal of an intermediate piece to disconnect the fluidical connection.

FIG. 7 is a schematic depiction of liquid transfer for removal of media from a cell culture container on the right to a waste container (potentially contaminated) on the left, where after welding a fresh waste tubing in the resulting gap from (see FIG. 6) the waste medium is transferred from right to left, sealed inside of the welds, cut within the seals and the intermediate piece is removed.

FIG. 8 is a schematic depiction of the sequential welding showing the weld progression from 1 through 4 transfers of liquid. An increasing length of fresh intermediate tubing is used in each successive weld in order to retain the length of tubing connected to the source and destination containers.

FIGS. 9A-9B include depictions of two variations of “nested welding” (panel A and panel B) in which fresh tubing is added to maintain an original tubing length and increase the number of possible connections.

FIG. 10 is an exemplary schematic depiction of the nested welding concept according to FIG. 9, panel A, showing weld locations and more tubing increments.

FIGS. 11A and 11B are schematic depictions of exemplary cell manufacturing processes, in which the automated sterile connection and liquid transfer methods and devices disclosed herein can be used, in accordance with some embodiments of the technology described herein. FIG. 11A: an exemplary process for cell culture. FIG. 11B: an exemplary process for manufacturing cells transduced with a viral vector.

FIG. 12 is a flowchart illustrating steps 402 to 416 in a method for automated liquid transfer between two containers, according to some embodiments.

FIG. 13. is an exemplary block diagram depicting the exemplary automated environment for liquid transfer between two containers.

### DETAILED DESCRIPTION

The present disclosure provides a method for the efficient combination of welding, liquid transfer, sealing, and separation operations, especially but not exclusively with the intention of enabling automation of some combination of these operations. The methods using such disclosed technology herein lead to at least the following advantageous outcomes:
A) High-throughput production of manufactured cells resulting from connection welds, liquid transfer, sealing, and cutting configured for use during various stages of the manufacturing process, e.g., for use during cell expansion and/or cell transduction. High-throughput production may also be achieved from operations disclosed herein that minimize the time needed to perform multiple liquid transfers. The operations disclosed herein may also be automated, thereby increasing production rates and productivity.
B) Reduced risk of cross contamination between containers and/or cell culture vessels resulting from connection welds configured for use with intermediate connectors that may be disposed of after a single use.

Accordingly, provided herein are connection welds for sterile connection, liquid transfer, sealing, disconnection and methods of use thereof. Such operations may be used for sterile connection and liquid transfer during cell manufacturing, e.g., manufacturing cell therapies such as T cells expressing a chimeric antigen receptor (CAR).

### I. Connection Welds for Sterile Connection and Liquid Transfer

The present disclosure provides a method for sterile automated liquid transfer between two containers includes: (a) placing a first tube and a second tube into a welding mount, wherein the first tube is connected to a first container and the second tube is connected to a second container, and (b) welding the first tube and the second tube to form a first sterile fluidical connection between the first container and the second container. The method also includes (c) transferring a liquid between the first container and the second container via the first sterile fluidical connection, (d) sealing and cutting the first fluidical connection between the first container and the second container to disconnect the first sterile fluidical connection, wherein steps (a)-(d) are operated automatically by a controller. Automatic operation of steps (a)-(d) includes the automated transition between each of the steps. Steps (a)-(d) may be repeated. The inventors provide for methods to automate all steps (a)-(d) including the loading of the welding head, which is important for multiple rounds of welding for example in the case of aliquoting larger amounts of biological products like cell suspensions or viral vectors into small aliquots in an automated and sterile method. Additionally, this disclosure combines automation of loading tubing, welding, fluid transfer, sealing, and sterile disconnection of tubing lines which enables automated culture of multiple cultures in serial fashion.

The method may be further applied for sterile automated liquid transfer with a third container by (e) placing a third tube into the welding mount, wherein the third tube is connected to a third container, (f) welding the first or second tube and the third tube to form a second sterile fluidical connection between the first container and the third container or between the second container and the third container, (g) transferring a liquid between the first container and the third container or between the second container and the third container via the second sterile fluidical connection, and (h) sealing the second fluidical connection between the connected first and third containers or between the connected second and third containers and cutting the seal to disconnect the second sterile fluidical connection, wherein steps (e)-(h) are operated automatically by a controller and wherein steps (a)-(h) and/or (e)-(h) are optionally repeated. Operation of steps (e)-(h) includes the automated transition between each of the steps.

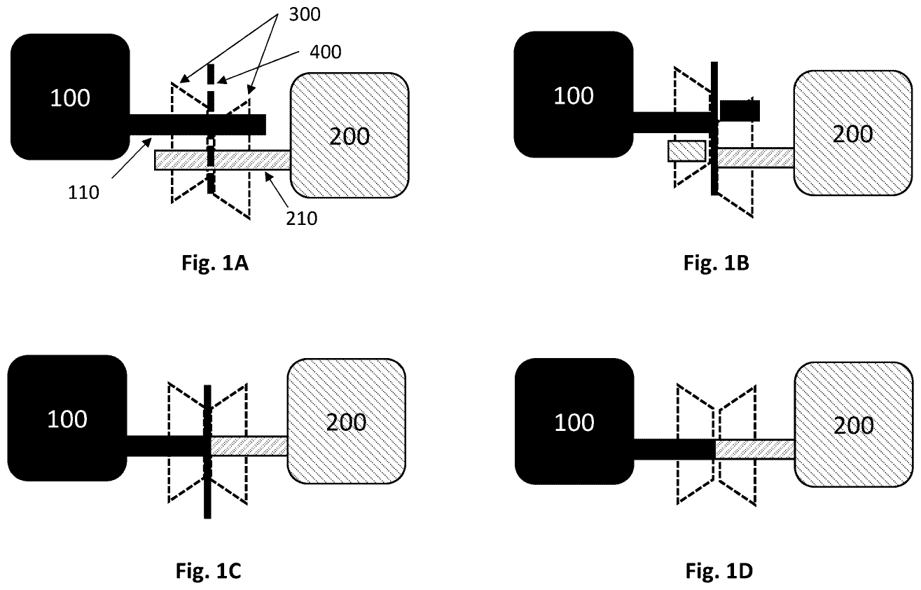

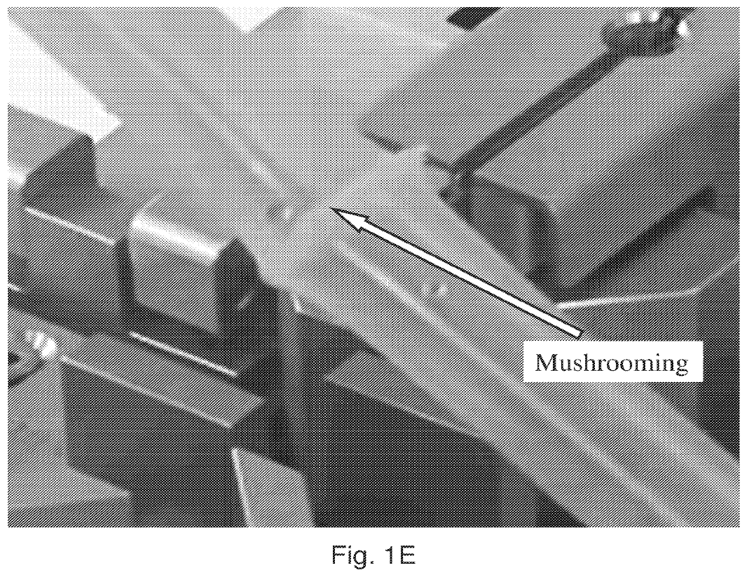

FIG. 1 shows an example of the current state of the art for tube welding in which a source container (100) and associated source container tubing (110) maybe be connected sterilely to a destination container (200) via the destination container tubing (210). In the state of the art, tubing (110 and 210) must be loaded into the weld mounts (300) manually, secured manually and welded by the Welding blade (400). (A) shows the initial position of the components with the welder blade above or below the tubes. (B) shows the sterile cutting of the tubes 110 and 210 through raising or lowering the heated welder blade (400) and displacing the cut ends of the tubes. (C) shows the alignment of the cut ends of 110 and 210 after movement of the weld mounts 300 still separated by the heated welder blade; cut ends have been discarded. (D) shows that after withdrawal (lowering or raising) of the heated welder blade, the ends of the tubes are annealed during cooling off while in close contact.

The state of the art is typically performed manually and cannot be easily automated as tube welds cannot be sealed and cut at the same position multiple times due to so-called mushrooming effect (see FIG. 1E), leading to a shortening of the connecting tube with each welding through deformation of the tubes when annealed together. Weld heads are not designed for automated loading, and tube welders are currently designed as stand-alone operations, independent from subsequent operations such as liquid transfer, sealing, and cutting or separation of tubing.

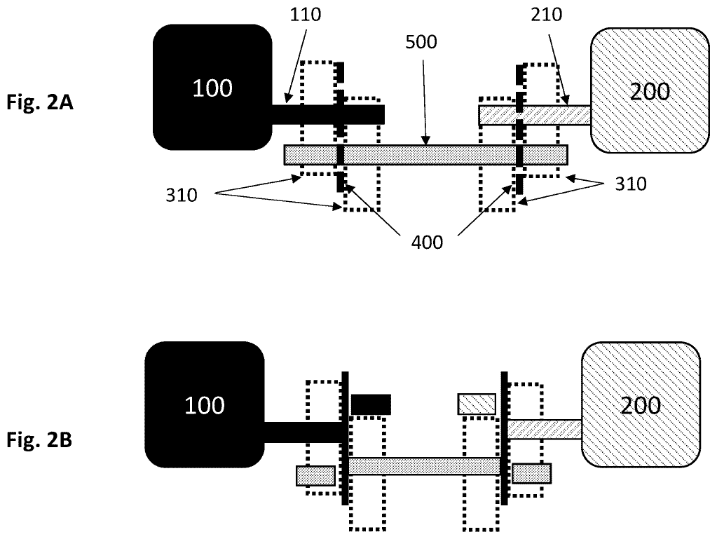

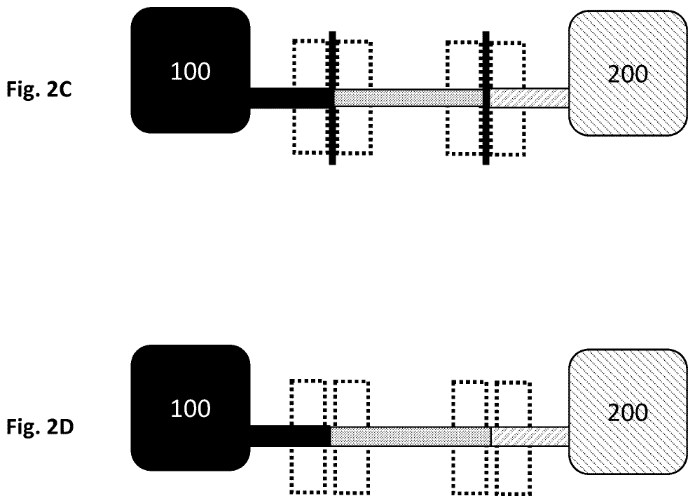

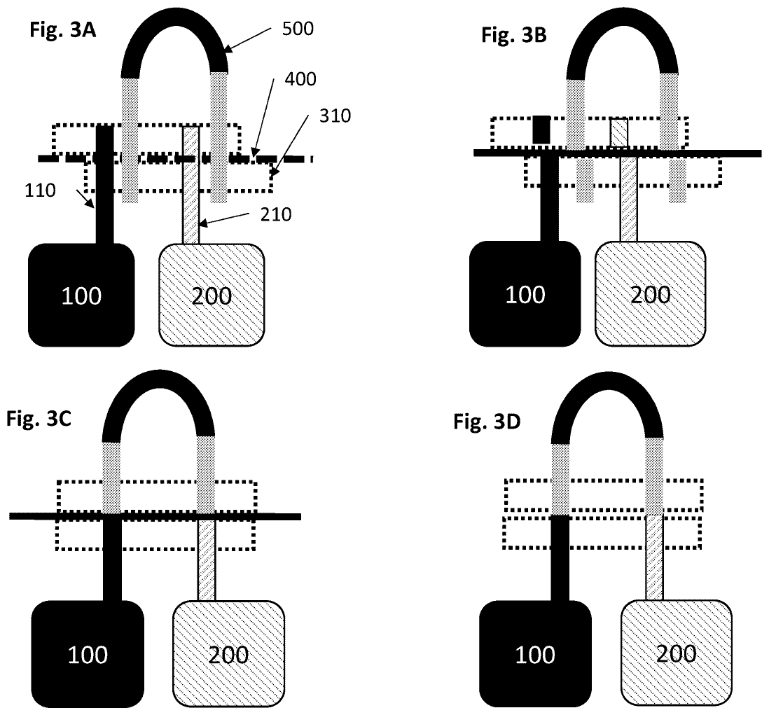

FIG. 2 shows tube welding with auto-loading weld mounts and an intermediate “spool piece” of tubing to prevent backflow contamination as improvements to current welding technology. The tubings and/or the intermediate/spool pieces are thermoplastic tubes well known in the art. Non-limiting examples include polythene, polythene copolymers, polypropylene, polyvinyl chloride, polyurethanes, nylon and polyester (such as polyethylene terephthalate, polyolefins) (see U.S. Pat. No. 4,619,642). Weld mounts (310) are designed to be automatically loaded and secured, and a second pair of weld mounts is positioned in proximity to the first, such that source container tubing (110) may be fluidically connected to destination container tubing (210) via a third spool piece of intermediate portion of tubing (500) in order to prevent backflow contamination between 110 and 210. The intermediate portion (500) may be a new piece of tubing or it may be formed from a longer piece of tubing that is sealed into the intermediate portion. The intermediate portion of tubing may be removable and/or disposable. Alternatively, or in addition to, the intermediate portion of tubing may be configured for liquid sampling, e.g., via sealing both ends after fluid transfer and excising the intermediate portion with fluid contained between the sealed ends. The two weld heads may optionally be synchronized. Non-limiting examples of liquid transfer include taking samples from a cell culture vessel, removing waste from a cell culture vessel, and transferring media and/or solution(s) from the container to a cell culture vessel. (A) shows the initial position of the components with the welder blade (400) above or below the tubes. (B) shows the sterile cutting of the tubes 110, 210 and the intermediate portion (500) through raising or lowering the heated welder blade (400) and displacing the cut ends of the tubes. (C) shows the alignment of the cut ends of 110 with one end of the intermediate portion 500 and 210 with the other end of the intermediate portion 500 after movement of the weld mounts 300 still separated by the heated welder blades; cut ends have been discarded. (D) shows that after withdrawal (lowering or raising) of the heated welder blade, the ends of the tubes are annealed during cooling off while in close contact with the inserted intermediate portion.

FIG. 3A-D shows two welds with one weld head and blade.

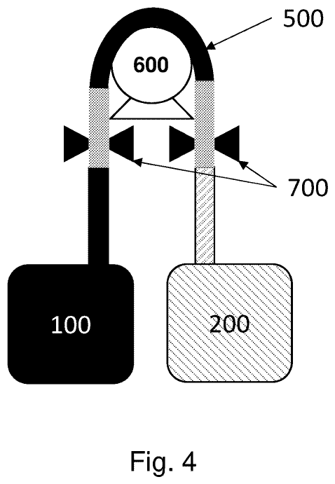

FIG. 4 shows a pump and pinch valve for fluid transfer and to prevent backflow contamination. A method is shown for fluid transfer between two containers using an intermediate tubing spool piece (500) with a pump (600) and optionally pinch valves (700) to regulate flow and minimize backflow contamination. Together with the sterile connection arrangement of FIG. 3, the fluid transfer configuration enables automatic sterile connection and liquid transfer. In some embodiments, the configuration may include a device for liquid transfer such a pump configured to transfer a liquid from the first container to the second container. Other examples for means to transfer a liquid from the first container to the second container are vacuum, a pressurizer and/or gravity. An interlock valve may be included to avoid back contamination between the first container and the second container. For example, a pump may be connected to the intermediate tubing, the fluid conduit of the container, and/or the fluid conduit of the cell culture vessel. Alternatively, or in addition to, the connection weld may use gravity to facilitate liquid transfer.

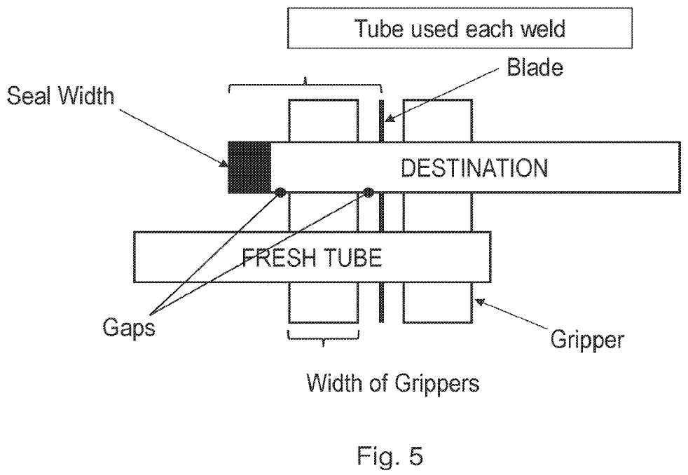

FIG. 5 shows a schematic configuration of a welding mount for a sterilized liquid transfer from a first container (e.g., coupled to a fresh tube) to a second container (e.g., coupled to a destination tube) from a top down perspective. To have a fixed tube length used with each weld, which depends on the seal width, the gaps and the width of the grippers of the weld mount. The fixed tube length for each welding process allows for automated multiple welding in fixed length increments. The lengths involved in a welding process are illustrated: a seal width, width of the grippers (aka weld mounts), a blade width, and gaps to allow for spacing tolerance for the grippers. Accordingly, each weld step involves a minimum length of tubing. Accordingly, the length of fresh tube added is desirably no less than the amount of tubing used for each weld, to maintain the tubing lengths coupled to each container fixed. Maintenance of tubing length is important to enable multiple connections to a single container, enable automation (i.e., location of tubing ends via automated grippers), and balanced weights for operations such as centrifugation.

The cutting device such as blade is simultaneously or subsequently used to make single or multiple cuts to the tubing. In some embodiments, the welding includes a cutting device wherein, the cutting device may be a wire, wafer, blade, knife, scissors, laser or alike. In some embodiments, the welding element includes using a single cutting device, preferably a blade to make two parallel cuts. In some embodiments, the welding element can include using multiple cutting devices, such as blades to make multiple parallel cuts. An initial position of the components may include having the welder blade above or below the tubes. In some embodiments, the welder blade is cold combined with a heating element. In some embodiments, the welder blade is heated to a pre-selected temperature. When the welder blade reaches the pre-selected temperature, it is raised or lowered (dependent on the positioning below or above the working surface) to contact and cut the first tubing, the second tubing, and the intermediate tubing. The tubing is aligned on the two parallel cuts: the first tubing is aligned with one end of the intermediate tubing, and the second tubing is aligned with the other end of the intermediate tubing. The blade is removed, and the heated tubing edges contact each other, completing the welding by annealing. The result is a fluidic coupling between the source container and the destination container. In some embodiments, the welding includes a laser beam (for e.g., Argon, CO₂ and YAG). In some embodiments, the pump is set to avoid any back contamination from the destination container to the source container. Activating the pump completes the fluid transfer from the source container to the destination container. The controller includes a memory circuit storing instructions and a processor circuit configured to execute the instructions, wherein, upon executing the instructions, the controller causes the welding element to fluidically couple the first tube and the second tube, optionally via a fresh piece of tubing of a selected length, and perform a specified liquid transfer.

The intermediate portion of tubing may be a new piece of tubing or it may be formed from a longer piece of tubing that is sealed into the intermediate piece. The intermediate piece of tubing may be removable and/or disposable. Alternatively, or in addition to, the intermediate piece of tubing may be configured for liquid sampling, e.g., via sealing both ends after fluid transfer and excising the intermediate portion with fluid contained between the sealed ends.

The source container or destination container for use in a connection weld disclosed herein may be any suitable shape or size, and any suitable material. For example, when receiving cells in the cell culture, the container may be a gas permeable material that permits diffusion of gases sufficient for cell viability. Such containers may be suitable for processing the cells, e.g., culturing and/or centrifuging the cells in the container. Alternatively, or in addition to, the container may be disposable to eliminate risks of contamination. Non-limiting examples of a container include a cell culture container (e.g., a cell culture bag), a destination bag, or a waste container. A destination bag may be used for either receiving the cell culture medium or the cells of the cell culture. A container for use in a connection weld disclosed herein may include a fluid conduit. The container may be a cell culture vessel of any suitable shape or size, and any suitable material. For example, the cell culture vessel may be disposable to eliminate risks of contamination. A non-limiting example of a cell culture vessel includes a cell culture bag.

Liquid may be transferred between the container and the cell culture vessel in either direction. In some embodiments, liquid, e.g., media, may be transferred from the (source) container to the destination container (cell culture vessel), e.g., for adding fresh culture medium. In some embodiments, liquid may be transferred to a (e.g., waste) container from the cell culture vessel, e.g., for removing culture medium. The container may be empty to receive the contents of the cell culture vessel or the container may include a solution to be transferred into the cell culture vessel.

### II. Methods for Connection Welding and Liquid Transfer

Also provided herein are methods for sterile connection via tube welding for multiple sequential weld connections to a single source and/or destination container, e.g., to enable cell therapy manufacturing and automation of cell therapy manufacturing operations. Methods disclosed herein involve sterile connection and liquid transfer between any source vessel and destination vessel. For example, when manufacturing cells, the source vessel may be a container including culture media and the destination vessel may be a cell culture vessel including a cell culture. In such instances, methods disclosed herein may be performed to transfer the culture media from the source vessel to the cell culture in the destination vessel.

One embodiment comprises a method for sterile automated liquid transfer between two containers, comprising: (a) placing a first tube and a second tube into a welding mount, wherein the first tube is connected to a first container and the second tube is connected to a second container; (b) welding the first tube and the second tube to form a first sterile fluidical connection between the first container and the second container; (c) transferring a liquid between the first container and the second container via the first sterile fluidical connection; (d) sealing the first fluidical connection between the first tube and the second tube to disconnect the first sterile fluidical connection, wherein steps (b) and (d) comprise welding, on one side of an existing weld, a selected length of a tubing, and adding new welds to the opposite side of the existing weld until the selected length of tubing is used up. Steps (a) to (d) can be operated automatically by a controller. Steps (a) to (d) are optionally repeated.

Further, in an embodiment, the method is further applied for sterile automated connection and liquid transfer with a third container comprising the further steps (e) placing a third tube into the welding mount, wherein the third tube is connected to a third container; (f) welding the first or second tube and the third tube to form a second sterile fluidical connection between the first container and the third container or between the second container and the third container; (g) transferring a liquid between the first container and the third container or between the second container and the third container via the second sterile fluidical connection; and (h) sealing the second fluidical connection between the connected first and third tubes or between the connected second and third tubes to disconnect the second sterile fluidical connection, wherein also steps (e) and (f) comprise welding, on one side of an existing weld, a selected length of a tubing, and adding new welds to the added tubing until the selected length of tubing is used up. Steps (a) to (d) or steps (e) to (h) are optionally repeated. All of the steps (a)-(d) are operated automatically by a controller, and wherein optionally all of the steps (e)-(h) are operated automatically by a controller. In some embodiments, welding the first tube to the second tube and/or the first or second tube to the third tube comprises welding a fresh portion of a tube of a pre-selected length in-between the first tube and the second tube, or in-between the first or second tube and the third tube, thereby connecting the first tube and second tube, or connecting the first or second tube to the third tube. In some embodiments, welding the first tube and the second tube, and/or welding the first or second tube and the third tube comprises (i) forming two separate sterile connections in the first tube and the second tube or in the first or second tube and the third tube with a heated welder blade, a laser or a cold blade combined with a heating element, a heated welder blade, and/or (ii) welding, on one side of an existing weld, a selected length of a tubing, and adding a new weld to the opposite side of the existing weld until the selected length of tubing is used up.

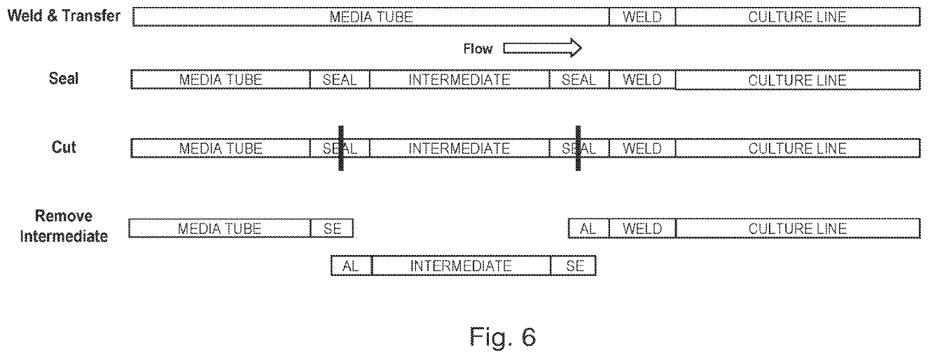

FIG. 6 to FIG. 10 are schematic depictions of an exemplary process for transferring a liquid and/or collecting a sample using tube welding, in accordance with some embodiments of the technology described herein. Nested welding as described herein can be applied to both welds to minimize source and destination bag tubing to use and maximize the number of welds per tubing length. Maximizing the number of welds per tubing length is important in order to enable multiple connections to a single container, e.g., a cell culture container for cell therapy manufacturing. Maintaining the tube length enables automation of operations involving containers, e.g. for cell therapy manufacturing, by allowing for a consistent location of the tubing end. Maintaining the tube length enables operations such as centrifugation by maintain an equivalent weight of tubing in order to maintain a balanced centrifugation load

A non-limiting example of a solution to be transferred from the container to the cell culture vessel is a culture medium for culturing cells in the cell culture vessel. An example of such transfer is shown in FIG. 6, where media may be transferred from a container on the left through a media tube, a weld and the culture line to a cell culture vessel on the right. Alternatively, or in addition to, the solution includes a nucleic acid for transducing cells grown in the cell culture vessel. Such nucleic acids may be delivered into cells using conventional technologies, e.g., transduction using reagents such as non-viral transduction (e.g., liposomes comprising non-viral vectors) or viral transduction (e.g., retroviral transduction such as lentiviral transduction). When the connection weld is being used to manufacturing cells expressing a chimeric antigen receptor (CAR), the solution may include a nucleic acid encoding the CAR.

By sealing and cutting within the fluidical path tubing twice in order to remove an intermediate piece, that then can be replaced by a piece of fresh tubing, the fluidical path can be reconstituted without changing the length of the tubing between the two containers, which allows for keeping the two containers at the same distance. This in turn allows for automation of the process. The displaced intermediate piece in this setting contains the transferred liquid which can be used to obtain a sample.

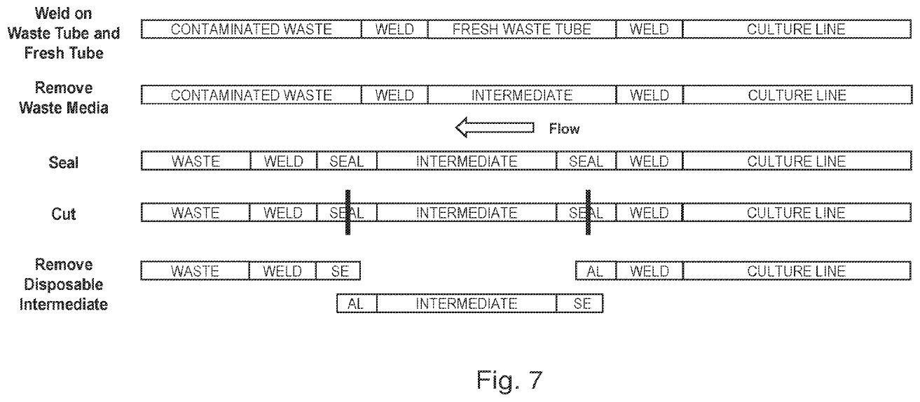

FIG. 7 shows tubing configured for transferring waste from a culture vessel to a container and the optional collecting a sample in the disposable intermediate. A piece of tubing welded between the waste line and culture line is sealed and cut to create the disposable intermediate.

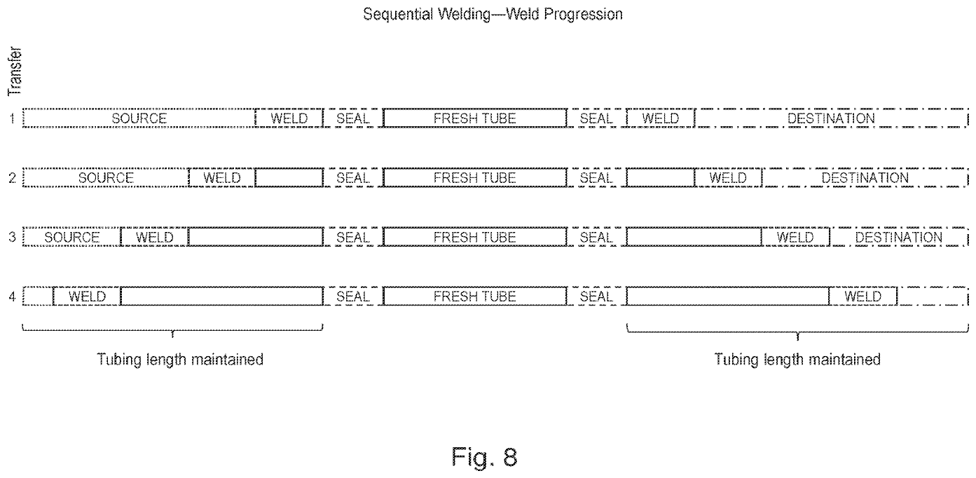

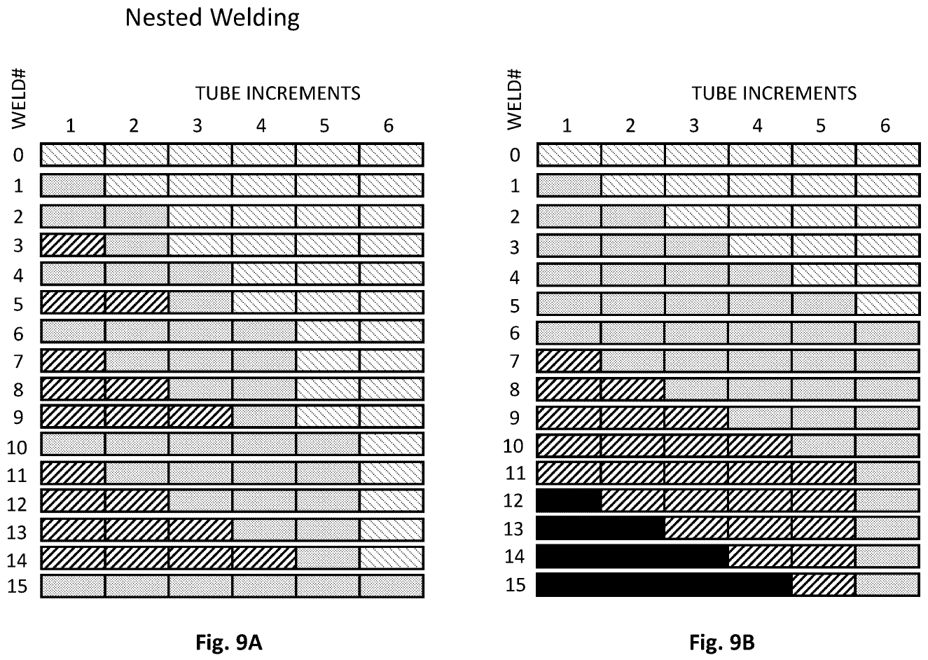

FIG. 8 illustrates tubing length of source and destination lines maintained by adding a piece of fresh tubing to the legacy tubing line with each successive weld. This is important for maintaining weight for centrifugation and standardizing line length to enable automation. Tubing length of source and destination lines is maintained by adding a piece of fresh tubing to the legacy tubing line with each successive weld. This is important for maintaining weight for centrifugation and standardizing line length to enable automation. Nested welding (see FIG. 9) can be applied to both welds to minimize source and destination bag tubing use/maximize the number of welds per tubing length.

Welding consumes a portion of the tubing (the “increment”) and tubing cannot be welded where it was previously welded (FIG. 6). Additional welds must be performed upstream or downstream of existing welds. The idea of nested welding is that it adds a length of fresh tubing to the length of the original tubing, the fresh tubing which itself can be welded multiple times, increasing the number of welds possible per increment of tubing. In some embodiments, the initial length of the tubing prior to the welding is maintained. In some embodiments, the initial length of the tube between the first container and the second container or the first container and the third container is substantially maintained. In some embodiments, welding the first tube and the second tube, or welding the first or second tube and the third tube comprises forming two separate sterile connections in the first tube and the second tube with a spool piece, or forming two separate sterile connections in the first or second tube and the third tube with a spool piece with a welder single blade, and wherein the spool piece prevents backflow contamination between the first container and the second container, or between the first or second container and the third container.

FIG. 9A illustrates one method of nested welding, wherein a minimal length of fresh tubing is added on when needed by welding to the right of existing welds, then new welds are added to the left of existing welds until the fresh tubing is used up. The cycle is then repeated.

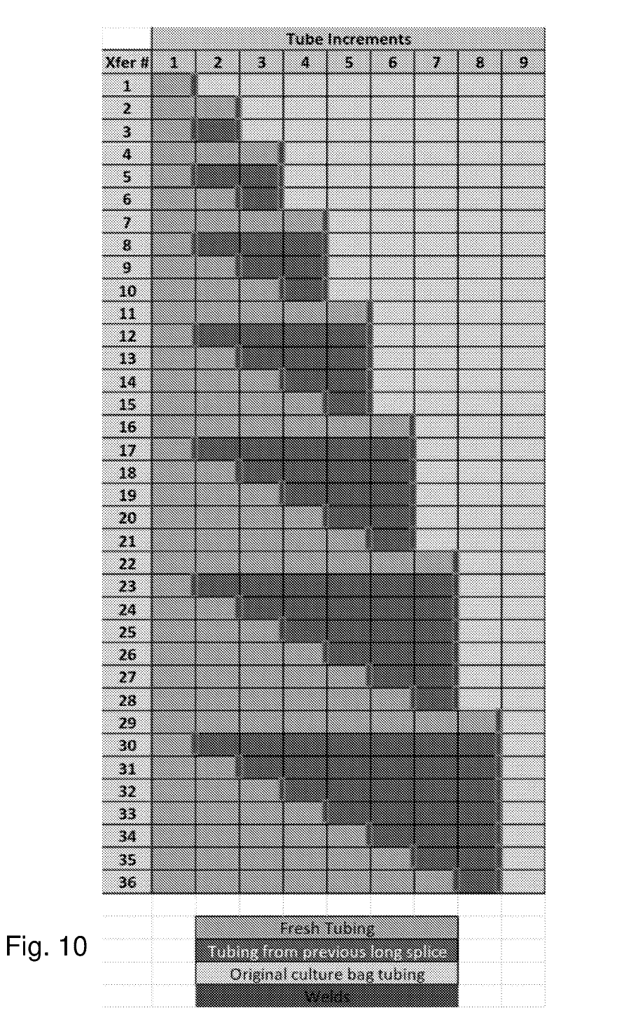

FIG. 9B illustrates a second method of nested welding, wherein welds are made from left to right until the entire length of original tubing is used up, then fresh tubing is welded on (as far right as possible). Fresh welds are made again from left to right until the added tubing is used up, then another fresh piece of tubing is welded on as far right as possible (but left of the prior weld, as that increment of tubing is no longer usable). FIG. 10 illustrates a nested welding process of FIG. 9A wherein by adding a length of fresh tubing at certain welding steps, a total of thirty six consecutive welds may be applied over a tube coupled to a container and having an initial length of nine times the pre-selected length. The length of tubing may be a multiple of a pre-selected length. The pre-selected length includes a minimum length of tubing desirable for the welding process (e.g., the gripper widths, the gap widths, the seal width, the mushrooming width, and the like). In some embodiments, the pre-selected length may include about 1 inch, or similar.

The grey indicates the length of fresh tubing welded in each step, and the purple indicates the length of tubing remaining from previous fresh tubing additions to the weld.

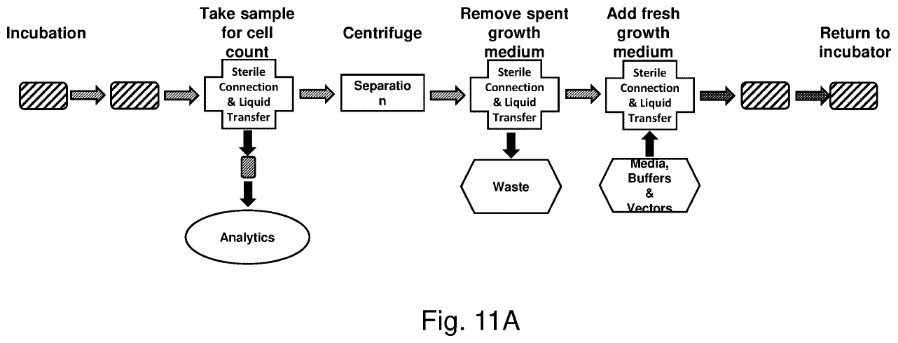

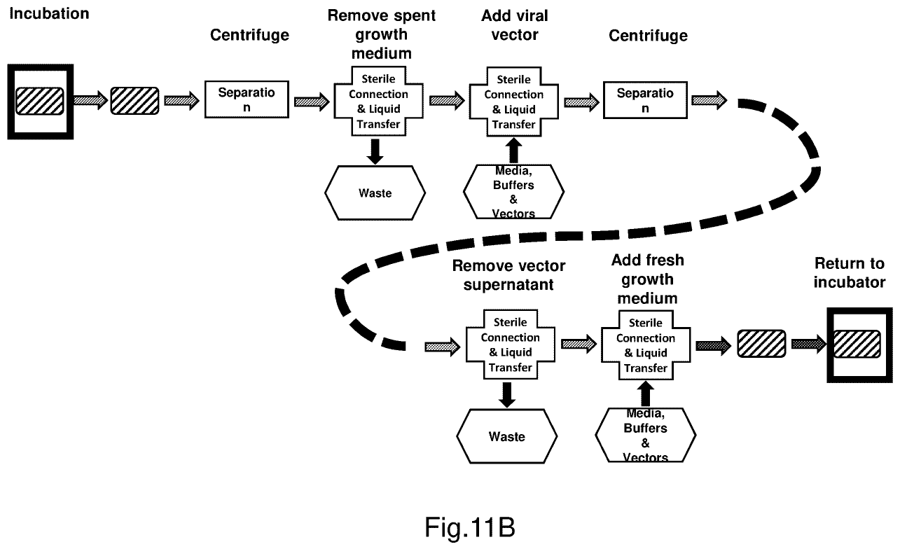

FIGS. 11A and B are schematic depictions of an exemplary process for manufacturing cells, in accordance with some embodiments of the technology described herein. FIG. 11A is a schematic depiction of an exemplary process for expanding a cell culture. Connection welds as disclosed herein may be used to perform various liquid transfers involved in the cell expansion process including taking a sample of the cells for analysis, removing spent growth medium, and adding fresh medium. A device capable of performing serial sterile connection and liquid transfer operations is shown as the blue plus in the workflow of a typical cell culture manufacturing operation of cell growth medium addition. External steps may be performed manually, or containers may be transferred by robotic arms or similar automated transfer devices. Automated loading of the weld mounts, liquid transfer, sealing, and cutting to disconnect containers are performed by the sterile connection and liquid transfer device. In this example, the sterile connection and liquid transfer device is used to first to remove medium (from the cell culture source bag to a destination waste medium bag) then to add liquid from the cell culture medium source bag to destination cell culture bag). Note that the waste medium destination bag and cell culture medium source bag may be serially connected to many cell culture bags (acting first as source bags, then as destination bags).

FIG. 11B is a schematic depiction of an exemplary process for transducing a cell culture. Connection welds as disclosed herein may be used to perform various liquid transfers involved in the cell transduction process including adding viral vector, removing vector supernatant, removing spent growth medium, and adding fresh medium. A device capable of performing serial sterile connection and liquid transfer operations is shown as the blue plus in the workflow of a typical CAR-T cell therapy manufacturing operation of viral vector transduction. External steps may be performed manually, or containers may be transferred by robotic arms or similar automated transfer devices. Automated loading of the weld mounts, liquid transfer, sealing, and cutting to disconnect containers are performed by the sterile connection and liquid transfer device. In this example, the sterile connection and liquid transfer device is used to first to remove medium (from the cell culture source bag to a destination waste medium bag) then to add liquid from the viral vector source bag to destination cell culture bag).

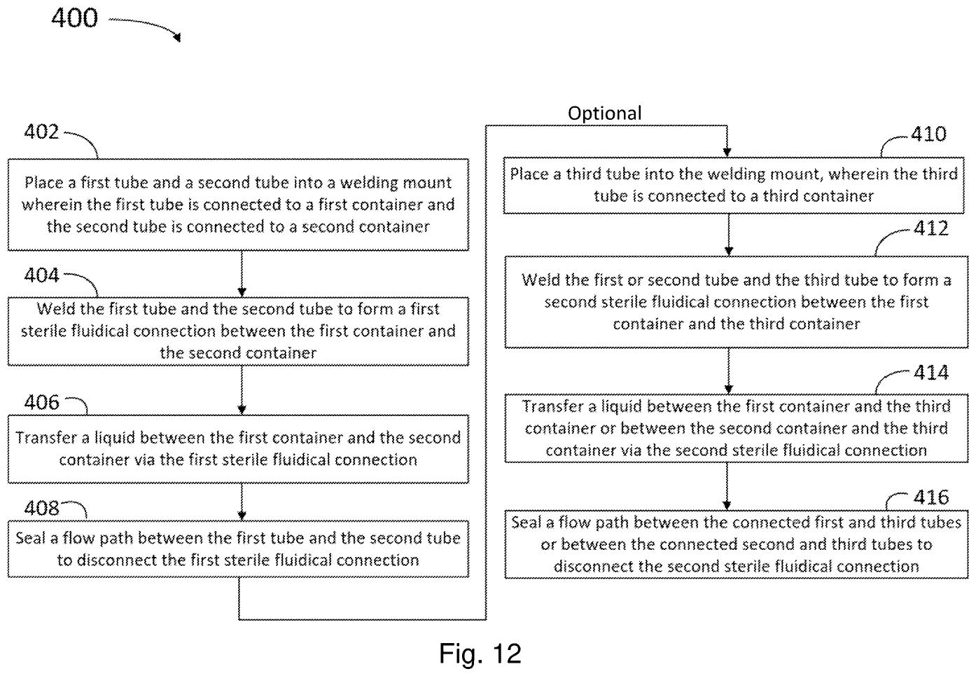

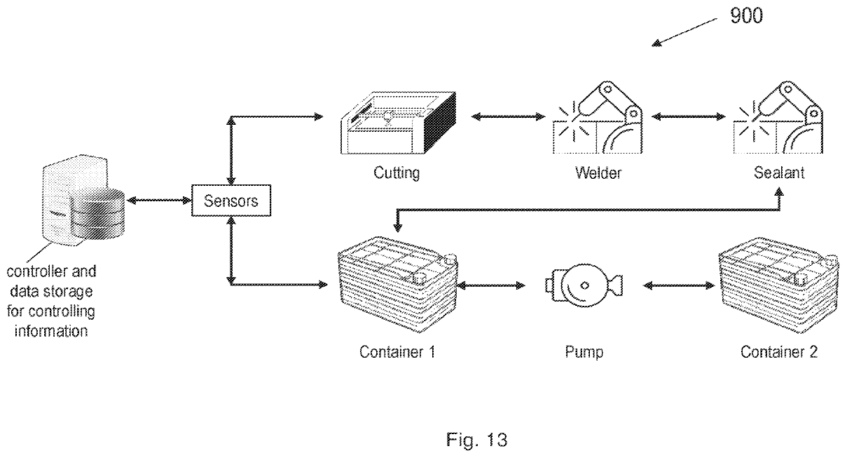

FIG. 12 is a flowchart illustrating steps in a method 400 for automated liquid transfer between two containers, according to some embodiments. In some embodiments, method 400 may be performed using a device as disclosed herein, including a welding mount configured to receive a first tube fluidically coupled to a first container and a second tube fluidically coupled to a second container. The method may involve further pairs of containers fluidically coupled to a tube, i.e. a third container fluidically coupled to a third tube, etc. The device may also include a welding element configured to fluidically couple the first tube with the second tube by welding and a pump configured to transfer a liquid from the first container to the second container and an interlock valve configured to avoid back contamination between the first container and the second container. In some embodiments, sealing a flow path between the first container and the second container, and/or between the first or second container and the third container comprises creating, for the first tube, two separate tubing seals, and the second tube with a single sealing head, and/or for the first or second tube, two separate tubing seals, and the third tube with a single sealing head. In some embodiments, the method further comprises moving the first container or second container to a connection interface. In some embodiments, the first container includes a cell culture medium and the second and/or third container includes cultured cells, wherein the cell culture medium is transferred from the first container to the cultured cells.

The device may also include a controller including a memory circuit storing instructions and a processor circuit configured to execute the instructions. Upon executing the instructions, the controller causes the welding element to be loaded with tubing, then to fluidically couple the first tube and the second tube, optionally via a fresh piece of tubing of a selected length, and perform steps in method 400, as follows.

Step 402 includes placing a first tube and a second tube and optionally a third container into a welding mount, optionally via automation, wherein the first tube is connected to a first container and the second tube is connected to a second container. In some embodiments, step 402 includes moving the first container or second container to a connection interface. In some embodiments, step 402 includes robotically loading the first tube, the second tube, and optionally the spool or intermediate piece of tubing within the welding mount.

Step 404 includes welding the first tube and the second tube to form a first sterile fluidical connection between the first container and the second container. In some embodiments, the spool or intermediate piece is used to prevent backflow contamination between the first container and the second container, and step 404 includes forming a first sterile fluidical connection by two separate sterile connections in the first tube and the second tube with the spool piece with a welder which can be a single blade.

Step 406 includes transferring a liquid between the first container and the second container via the first sterile fluidical connection. Transfer may be made through a pump, vacuum, pressure or gravity.

Step 408 includes sealing by at least one seal (e.g., by two seals) a flow path between the first tube and the second tube and cutting the seal(s) to disconnect the first sterile fluidical connection. Sealing may be performed with existing heat sealing and/or mechanical crimping techniques. Optionally, a single heat sealing head may be designed and positioned to create two separate seals, e.g. similar to the two welds of FIG. 2. Optionally the cutting of two separate seals may be performed with a single blade configured similar to the tube welding blade of FIG. 2. Sealing and cutting may be automated.

Step 410 includes placing a third tube into the welding mount, wherein the third tube is connected to a third container. Step 410 includes robotically loading the third tube with the third container.

Step 412 includes welding the first or second tube and the third tube to form a second sterile fluidical connection between the first container and the third container or between the second container and the third container. In some embodiments, at least one of welding steps 404 or 412 includes welding a fresh portion of a tube of a pre-selected length connecting the first tube and second tube, or connecting the first or second tube to the third tube. In some embodiments, at least one of welding steps 404 or 412 includes forming two separate sterile connections in the first tube and the second tube or in the first or second tube and the third tube with a welder single blade. In an embodiment the welder single blade is a heated welder blade. Alternatively, a laser or a cold blade combined with a heating element can be used (see U.S. Pat. No. 6,913,056). In some embodiments, at least one of welding steps 404 or 412 includes welding, on one side of an existing weld, a selected length of a tubing, and adding a new weld to the opposite side of the existing weld until the selected length of tubing is used up. In some embodiments, at least one of welding steps 404 or 412 includes welding, on one side of an existing weld, a selected length of a tubing, and adding a new weld to the opposite side of the existing weld until the selected length of tubing is used up. In some embodiments, at least one of welding steps 404 or 412 that includes welding the selected length of tubing includes welding a piece of fresh tubing having a length equal to the selected length of tubing to the third tubing. In some embodiments, the selected length of a tubing includes a multiple of a first length, the first length including a mushrooming length, a gripper width, a gap, and a seal width, and at least one of welding steps 404 or 412 that includes welding the selected length of tubing includes slicing a piece of the third tubing having the first length. In some embodiments, at least one of welding steps 404 or 412 includes determining the selected length of tubing so that a length of the second tubing is equal to a length of the third tubing after completing the welding. In some embodiments, the method further comprises determining the selected length of tubing in any of the welding steps based on the existing length of unwelded tubing and a desired length for a tubing coupled to a container for liquid transfer in the latest cycle. In some embodiments, multiple cycles may be repeated.

Step 414 includes transferring a liquid between the first container and the third container or between the second container and the third container via the second sterile fluidical connection. In some embodiments, at least one of step 406 or 414 includes interlocking one or more pinch valves with a peristaltic pump and slightly rotating the peristaltic pump to create a positive or a negative pressure in one of the first tube or the second tube prior to releasing the one or more pinch valves. In some embodiments, at least one of step 406 or 414 includes rotating a peristaltic pump to cause a positive or a negative pressure in one of the first tube or the second tube. In some embodiments, at least one of step 406 or 414 includes rotating a peristaltic pump between two interlocking valves prior to activating the interlocking valves. In some embodiments, at least one of step 406 or 414 includes transferring a solution including a nucleic acid or a viral particle including such, to a cell culture for transducing a plurality of cells therein, optionally wherein the nucleic acid encodes a coding of a chimeric receptor. In some embodiments, the cell culture includes immune cells, which optionally are T cells. In some embodiments, the immune cells express the chimeric receptor (e.g., a CAR).

In some embodiments, at least one of step 406 or 414 includes transferring a culture medium or a plurality of cells into the designation bag from a cell culture vessel. In some embodiments, the second container is a cell culture vessel, and step 414 includes transferring liquid between the cell culture vessel and the third container. In some embodiments, at least one of step 406 or 414 includes pumping the liquid from the first container to the second container or pumping the liquid from the first or second container to the third container.

Step 416 includes sealing a flow path between the connected first and third tubes or between the connected second and third tubes to disconnect the second sterile fluidical connection. In some embodiments, at least one of steps 408 and 416 includes creating, for the first tube, two separate tubing seals, and the second tube with a single sealing head, and/or for the first or second tube, two separate tubing seals, and the third tube with a single sealing head.

In some embodiments, method 400 may be repeated multiple times, being automatically executed by the controller. Method 400 may be used for multiple containers connected to a tubing—in a tree or manifold or similar configuration, e.g., multiple cell culture vessels connected to a tubing on one side and multiple containers connected to a tubing on the other side, e.g. containing medium, solutions for cell manipulation and a waste container, Containers of choice may be robotically placed into the weld mounts. In an embodiment, loading the first tube, the second tube, and optionally the third tube within the welding mount comprises robotically loading the first tube, the second tube, and the third tube within the welding mount.

Any number of sterile connections and liquid transfers may be performed by repeating the moving step, the connecting step, and the transferring step until the desired number of sterile connections and liquid transfers has been performed. The desired number of sterile connections and liquid transfers can be performed using a single connection weld or multiple connection welds. A single connection weld may be used to perform a single sterile connection and liquid transfer or multiple sterile connections and liquid transfers. Alternatively, or in addition to, multiple sterile connections and liquid transfers may be performed using multiple connection welds.

In some embodiments, the sterile connection and liquid transfer methods and devices disclosed herein can be used for serial sterile liquid transfers in various manners. For example, a liquid (e.g., cell culture medium) can be transferred between contains in a one-to-many manner using any of the methods and devices disclosed herein, e.g., from on source container to multiple destination containers (e.g., containing cells) in a sequential manner via serial connections and disconnections. Alternatively, a liquid can be transferred in a many-to-one manner between containers, e.g., from multiple source containers (e.g., containing cell culture medium, viral vectors, growth factors, etc.) to one destination container in a sequential manner via serial connections and disconnections. In another example, a liquid can be transferred in a many-to-many manner between containers, e.g., from multiple source containers to multiple destination container in a sequential manner via serial connections and disconnections.

It should be appreciated that methods disclosed herein may be performed manually and/or by one or more robotic devices such as robots or robotic arms. For example, the step(s) of moving the cell culture vessel to the connection weld(s) or vice versa may be performed by one or more robotic devices operated by a controller. In another example, the step(s) of connecting the cell culture vessel to the container(s) may be performed by one or more robotic devices. In yet another example, the cell culture vessel including a cell culture may be taken from an incubator arranged to house a plurality of cell culture vessels by one or more robotic devices.

A sterile connection and liquid transfer may be performed before and/or after processing of the cell culture in the cell culture vessel. For example, the cell culture vessel including the cell culture may be centrifuged and/or mixed prior to performing a sterile connection and liquid transfer. In another example, the cell culture vessel including the cell culture may be centrifuged and/or mixed after performing a sterile connection and liquid transfer. In yet another example, the cell culture vessel including the cell culture may be centrifuged and/or mixed both before and/or after performing a sterile connection and liquid transfer.

Methods disclosed herein encompass any moving of the cell culture vessel and the connection weld such that a sterile connection and liquid transfer may be performed. Accordingly, the moving step may involve moving the cell culture vessel to the connection weld or moving the connection weld to the cell culture vessel. Alternatively, or in addition to, the moving step may involve moving both the cell culture vessel and the connection weld.

Liquid transfer may be achieved using any suitable method for transferring liquids, e.g., transfer via gravity or a device such as a pump, vacuum, or pressurizer.

Connection welds and methods for sterile connection and liquid transfer described herein can be used for manufacturing cells, e.g., manufacturing immune cells expressing a chimeric receptor. Manufacturing cells may include culturing cells, expanding cells, or transducing cells. Manufacturing cells may involve any number of connection welds used to perform any number of sterile connections and liquid transfers.

Accordingly, methods disclosed herein may involve transferring cells and/or reagents for manufacturing cells, e.g., cell culture medium for growing cells and/or solutions including nucleic acids for transducing cells. For example, when manufacturing cells expressing a chimeric receptor, a solution including a nucleic acid coding for the chimeric receptor may be transferred from a container to a cell culture vessel for transducing the cells therein.

In some embodiments, multiple source containers may be connected sequentially to a single destination container (e.g., for adding/removing media and solutions to a single cell culture vessel). In some embodiments, multiple destination containers may be connected sequentially to a single source container (e.g., for adding media to multiple cell culture vessels). In some embodiment, multiple source containers may be connected sequentially to multiple destination containers (e.g., for adding/removing media and solutions to multiple cell culture vessels).

It should be appreciated that various embodiments of the present disclosure may be formed with one or more of the above-described features. The above aspects and features of the disclosure may be employed in any suitable combination as the present disclosure is not limited in this respect. It should be appreciated that the drawings illustrate various components and features which may be incorporated into various embodiments of the present disclosure. For simplification, some of the drawings may illustrate more than one optional feature or component. However, the present disclosure is not limited to the specific embodiments disclosed in the drawings. It should be recognized that the present disclosure encompasses embodiments which may include only a portion of the components illustrated in anyone drawing figure, and/or may also encompass embodiments combining components illustrated in different figures.

Such methods may use multiple containers to transfer cells and/or reagents into a cell culture vessel for manufacturing cells, e.g., for transducing cells. For example, the first container may include a cell culture medium for culturing cells, the second container may include a solution including a nucleic acid for transducing the cells, and the third container may be a destination bag for receiving either the cell culture medium or the cells.

Methods disclosed herein may also involve collecting the cells. For example, methods disclosed herein may result in collection of the cells in a container such as a destination bag. As such, methods disclosed herein may further include centrifuging a cell culture to obtain the collection of cells. Cells may be collected at any point during the manufacturing process, e.g., when transferring cells to a larger cell culture vessel during cell expansion or when harvesting cells for downstream processing or therapeutic use. Accordingly, cells may be collected in any one of the containers (e.g., the first, second, or third containers) used when performing multiple sterile connections and liquid transfers.

### III. Device

Also provide herein is a device, comprising a welding mount configured to receive a first tube fluidically coupled to a first container and a second tube fluidically coupled to a second container, further comprising a welding element configured to fluidically couple the first tube with the second tube by welding, a pump configured to transfer a liquid from the first container to the second container, optionally an interlock valve configured to avoid back contamination between the first container and the second container, and a controller comprising a memory circuit storing instructions and a processor circuit configured to execute the instructions, wherein, upon executing the instructions, the controller causes the welding element to fluidically couple the first tube and the second tube, optionally via a fresh piece of tubing/spool piece of a selected length. The device can be configured for use in bioprocessing of cells.

In some embodiments, the controller automatically operates the loading of the welding mount, welding, transfer of fluid, sealing and disconnection. In an embodiment, the welding element comprises clamps and/or holders and cutting device, a heated welder blade, a laser or a cold blade combined with a heating element. A heated welder blade is well known in the art and may be used for cutting the first tube and the second tube cutting/melting the tube, cut tube ends can then be moved along the heated welder blade to position for alignment with tube ending intended for coupling, and by removal of the heated welder blade the aligned endings are fused and generate a fluidical connection.

In one embodiment, the welding mount is designed to work with automation to load the tubing. In another embodiment, the device may further comprise a welding mount configured to receive a piece of fresh tubing/spool piece of a selected length, and a single or plurality of welding elements configured to fluidically couple the first tube with one side of the piece of fresh tubing and the other side of the piece of fresh tubing with the second tube by welding; the controller is configured to automatically operate the device with its elements (a) to (c) and/or (e)-(f).

In one embodiment, the device comprises a sealing device and cutting device configured to seal and cut the flow path. The cutting device may be a wire, wafer, blade, knife, scissors, laser or alike. In some embodiments, the welding element includes a single cutting device, a blade to make two parallel cuts. In some embodiments, the welding element includes multiple cutting devices, blades to make multiple parallel cuts. An initial position of the components may include having the welder blade above or below the mounts for the tubes. In some embodiments, the welder blade is cold combined with a heating element. In some embodiments, the welder blade can be heated to a pre-selected temperature. The heated welder blade may be controlled that once it reaches the pre-selected temperature, it is raised or lowered (dependent on the positioning below or above the working surface) to contact and cut the first tubing, the second tubing, and the intermediate tubing. In some instances, wherein the cutting device comprises a cold blade, the welding element and sealing device is different. In some instances, wherein the cutting device comprises a hot blade, the welding element and sealing device may be the same different. In some embodiments, the welding element includes a laser beam (for e.g., Argon, CO₂ and YAG).

In one embodiment, the device comprises a peristaltic pump between the first tube and the second tube to prevent backflow between the first container and the second container.

In one embodiment, the device comprises an interlocking pinch valve between the first tube and the second tube to prevent backflow between the first container and the second container. In some embodiments, the controller can be configured to activate the welding element over the first tube and the second tube.

The controller may be programmed to align the tubing on the two parallel cuts: the first tubing is aligned with one end of the intermediate tubing, and the second tubing is aligned with the other end of the intermediate tubing. The controller may be programmed to then remove the blade so that the heated tubing edges contact each other in order to complete the welding by annealing. In some embodiments, the controller is configured to automatically activate the welding element over the first tube and the second tube. In another embodiment wherein the controller is configured to seal a flow path between the first container and the second container.

In some instances, a controller may be programmed to perform the steps automatically. In some embodiments, the controller comprises a memory circuit and a processor circuit, the memory circuit storing instructions which, when executed by the processor circuit, cause preceding embodiments to be performed automatically. As depicted in FIG. 13, the system 900 can comprise a computing device (controller) which can be in communication with a data storage device. In an embodiment, the data storage device can store system data and at least one operating parameter. The data storage can be in the same location as the controller or at an offsite location wherein the controller is in telecommunication with the data storage system.

The system 900 can further comprise a plurality of sensors, the sensors can comprise measuring devices that are configured to provide data to the controller regarding the operation of each component within the system. The sensors displayed in the system can include but not limited to: position sensors, pressure sensors, optical sensors, temperature sensors, force sensors, vibration sensors, piezo sensors, fluid property sensors (to measure e.g., viscosity, pH level, CO₂ level, etc.), time sensors and/or humidity sensors located at the system components. The system can comprise these sensors to provide data to the controller to initiate and maintain operation of the system. For example, a sensor located at the cutting device can be a length measuring sensor to determine the length of the tube to be cut. In a further example, the measuring device can be an ultrasonic, velocimeter or laser measuring device. The cutting device sensor can also include a temperature measuring sensor. The cutting device can also comprise force and motion sensors, or optical sensors, such as when a gripper is used to grab, push, and/or pull tubing into position to be cut. In the case that a laser is used to cut the tubing, the temperature sensor can provide operational input to the controller that the tubing is being cut under the defined operation conditions in accordance with machine instructions. Similar to the cutting device, the welding device and sealant device can include sensors that monitor the operational activity of the welder and sealer. In the case of the sealer or welder, the sensors can also include a pressure sensor to determine if the pressure exerted on the walls by the tubing is consistent with operation parameters. The sensors associated with the system at the cell containers and/or pump can comprise flow rate sensors to determine whether fluid is flowing at the defined level and speed between the containers. The data received from the sensors located at the various components of the systems provided data to automate a continuous feedback loop that permits the controller to maintain and adjust the operation of all components of the system.

In an exemplary embodiment, the controller operates the tube welding using a spool piece according to the algorithm as depicted in FIG. 10 by saving the number of weldings, directing position of the next welding position using length/position sensors according to tube increments and the number of previous weldings as provided by FIG. 10, thereby allowing for up to 36 welding steps for one container. According to this algorithm, tube lengths are defined/maintained so that the grippers of a robot are enabled to be properly controlled by the controller grip the end of the tubing at the respective round of welding, which enables automation.

In a one embodiment, the controller which controls the welding, fluid transfer, sealing and cutting of tubing between culture vessels is part of a device for sterile connection and liquid transfer which can communicate with other devices in an automated facility for cell culture. In another example of such a facility, a typical workflow involving the method for sterile automated liquid transfer may be the workflows depicted in FIG. 11A and FIG. 11B. In FIG. 11A a container, e.g. a cell culture vessel containing a tissue culture, is incubated in an incubator at suitable conditions. The incubator is arranged to house multiple cell culture vessels, each being configured for moving in and out of the incubator independently by a robot. A controller is programmed to move individual containers in and out of the incubator by the robot depending on data provided by sensors or at pre-defined timepoints. Time values and values of sensors are stored for each individual container/cell culture vessel by the controller. The controller is programmed to conduct specific activities at specific timepoint and/or when specific monitored values that are measured for each container within the incubator are achieved.

In such a facility, for example, at a defined timepoint, a controller directs a robot to move a specific container comprising a tissue culture (cell container), which comprises a tube, due for such time point from the incubator into the device according to the disclosure so that the tube of the cell container is placed into the welding mount of the sterile connection and liquid transfer device. The robot further puts the tube connected to a device for measuring the cell density/cell count into the welding mount as well as a fresh tubing/spool piece. Next, the controller conducts the claimed method of sterile automated liquid transfer and directs a pump to pump a small volume of cell suspension from the cell container into the device for measuring the cell count, which determines the cell count and has an interface with the controller to transmit the cell count value to the controller. The controller compares the cell count value a with predefined value/range. Next, the controller is interrupting the fluidical connection by the sealing device and cutting the fluidical connection. If the measured value is too low, the controller controls the robot to return the cell container to the incubator and reschedules the cell container for the next manipulation step according to a preset algorithm. If the measured value is at the defined value or higher, the controller controls the robot to place the cell container into a centrifuge and controls the centrifuge to centrifuge the cells at a given speed and time. After the centrifugation, the controller controls the robot to move the cell container back into the welding mount of the device and puts the tube connected to a waste container in the welding mount. Again, the controller conducts the claimed method for sterile automated liquid transfer to connect the cell container through a spool piece to the tube of the waste container, and remove through a pump a predefined volume of spent growth medium from the cell container into the waste container. Then, the controller directs the cutting of the fluidical connection and sealing of the ends of the tubes, directs the robot to put the waste container back to a predefined position and to bring and position in the mount of the device another tube connected to a medium container, and conducts another method for sterile automated liquid transfer of the tube of the container through a spool piece to another tube connected to the medium container having fresh growth medium (or other liquids for manipulation of the cells such as vector). The controller directs a pump to pump a defined volume of medium into the container, cuts the fluidical connection and seals the ends of the tubes (per FIG. 11A). The controller may repeat the method for sterile automated liquid transfer for further steps of manipulation of the cells in the container (e.g., addition of specific feeds, cytokines, or the process of FIG. 11B) according to programs predefined or by values transmitter from sensors to the controller programmed to undertake specific cell manipulations for specific values. Once all of the defined cell manipulation steps have been completed, the controller directs the robot to remove the cell container (with sealed tubing) from the mount of the device and return it to the incubator at a predefined position or at a random empty position, which is then stored by the controller.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents, and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “including” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein in the specification and in the claims, the term “connected” is defined as attached, whether directly or indirectly through intervening components, and is not necessarily limited to physical connections. The connection can be such that the objects are permanently connected or releasably connected.

It should also be understood that, unless clearly indicated to the contrary, in any methods claimed herein that include more than one step or act, the order of the steps or acts of the method is not necessarily limited to the order in which the steps or acts of the method are recited.

The disclosure is also described by the following items:

1. A method for sterile automated liquid transfer, comprising:
(a) placing a first tube and a second tube into a welding mount, wherein the first tube is connected to a first container and the second tube is connected to a second container;
(b) welding the first tube and the second tube to form a first sterile fluidical connection between the first container and the second container;
(c) transferring a liquid between the first container and the second container via the first sterile fluidical connection;
(d) sealing and cutting the first fluidical connection between the first container and the second container to disconnect the first sterile fluidical connection;
   wherein at least one of the steps (a)-(d) is operated automatically by a controller.

2. The method of item 1, wherein the method further comprises:
(e) placing a third tube into the welding mount, wherein the third tube is connected to the third container;
(f) welding the first or second tube and the third tube to form a second sterile fluidical connection between the first container and the third container or between the second container and the third container;
(g) transferring a liquid between the first container and the third container or between the second container and the third container via the second sterile fluidical connection; and
(h) sealing the second fluidical connection between the connected first and third containers or between the connected second and third containers to disconnect the second sterile fluidical connection;
   wherein steps (e)-(h) are operated automatically by a controller and
   wherein steps (a)-(h) or (e)-(h) are optionally repeated.

3. The method of any one of the preceding items, wherein welding the first tube to the second tube and/or the first or second tube to the third tube comprises welding a fresh portion of a tube of a pre-selected length in-between the first tube and the second tube, or in-between the first or second tube and the third tube, thereby connecting the first tube and second tube, or connecting the first or second tube to the third tube.

4. The method of any of the preceding items, wherein welding the first tube and the second tube, and/or welding the first or second tube and the third tube comprises forming two separate sterile connections in the first tube and the second tube or in the first or second tube and the third tube with a welder single blade.

5. The method of any of the preceding items, wherein in step (b) and/or step (f), welding the first tube and the second tube, or welding the first or second tube and the third tube comprises forming two separate sterile connections in the first tube and the second tube with a spool piece, or forming two separate sterile connections in the first or second tube and the third tube with a spool piece with a welder single blade, and wherein the spool piece prevents backflow contamination between the first container and the second container, or between the first or second container and the third container.

6. The method of any one of the preceding items, wherein the controller comprises a memory circuit and a processor circuit, the memory circuit storing instructions which, when executed by the processor circuit, cause steps (a)-(d) and/or (e)-(h) to be performed automatically.

7. The method of any one of the preceding items, wherein welding the first tube and the second tube and/or the first or second tube and the third tube comprises welding, on one side of an existing weld, a selected length of a tubing, and adding a new weld to the opposite side of the existing weld until the selected length of tubing is used up.

8. The method of any one of the preceding items, wherein sealing a flow path between the first container and the second container, and/or between the first or second container and the third container comprises creating, for the first tube, two separate tubing seals, and the second tube with a single sealing head, and/or for the first or second tube, two separate tubing seals, and the third tube with a single sealing head.

9. The method of any one of the preceding items, wherein step (a) comprises moving the first container or second container to a connection interface.

10. The method of any one of the preceding items, wherein the first container comprises a cell culture medium and the second and/or third container comprises cultured cells, and wherein the cell culture medium is transferred from the first container to the cultured cells.

11. The method of any one of the preceding items, wherein transferring a liquid between the first container and the second container and/or between the first or second container to the third container comprises interlocking one or more pinch valves and/or a peristaltic pumps.

12. The method of any one of the preceding items, wherein transferring a liquid between the first container and the second container and/or between the first or second container and the third container comprises slightly rotating the peristaltic pump to create a positive or a negative pressure in one of the first tube or the second tube prior to releasing the one or more pinch valves to cause a positive or a negative pressure in one of the first tube or the second tube.

13. The method of any one of the preceding items, wherein transferring a liquid between the first container and the second container and/or between the first or second container and the third container comprises rotating a peristaltic pump between two interlocking valves prior to activating the interlocking valves.

14. The method of any one of the preceding items, wherein transferring a liquid between the first container and the second container and/or between the first or second container and the third container comprises transferring a solution including a nucleic acid or a viral particle comprising such, to a cell culture for transducing a plurality of cells therein, optionally wherein the nucleic acid encodes a chimeric receptor.

15. The method of item 14, wherein the cell culture comprises immune cells, which optionally are T cells.

16. The method of item 15, wherein the immune cells express the chimeric receptor.

17. The method of any one of the preceding items, wherein the first container is a cell culture vessel, the second and/or third container is a designation bag, and transferring a liquid between the first container and the second container comprises transferring a culture medium or a plurality of cells into the designation bag from a cell culture vessel.

18. The method of any one of the preceding items, wherein the second container is a cell culture vessel, and wherein the method further comprises transferring liquid between the cell culture vessel and the third container.

19. The method of any one of the preceding items, wherein loading the first tube, the second tube, and optionally the third tube within the welding mount comprises robotically loading the first tube, the second tube, and the third tube within the welding mount.

20. The method of any one of the preceding items, wherein transferring a liquid between the first container and the second container and/or between the first or second container and the third container comprises pumping the liquid from the first container to the second container or pumping the liquid from the first or second container to the third container.

21. A method for sterile automated connection and liquid, comprising:
(a) placing a first tube and a second tube into a welding mount, wherein the first tube is connected to a first container and the second tube is connected to a second container;
(b) welding the first tube and the second tube to form a first sterile fluidical connection between the first container and the second container;
(c) transferring a liquid between the first container and the second container via the first sterile fluidical connection;
(d) sealing the first fluidical connection between the first container and the second container to disconnect the first sterile fluidical connection;
   wherein steps (b) and (d) comprise welding, on one side of an existing weld, a selected length of a tubing, and adding new welds to the added tubing until the selected length of tubing is used up.

22. The method of item 21, wherein the method further comprises:
(e) placing a third tube into the welding mount, wherein the third tube is connected to a third container;
(f) welding the first or second tube and the third tube to form a second sterile fluidical connection between the first container and the third container or between the second container and the third container;
(g) transferring a liquid between the first container and the third container or between the second container and the third container via the second sterile fluidical connection; and
(h) sealing the second fluidical connection between the connected first and third tubes or between the connected second and third tubes and cutting the seal to disconnect the second sterile fluidical connection,
   wherein steps (e) and (f) comprise welding, on one side of an existing weld, a selected length of a tubing, and adding new welds to the added tubing until the selected length of tubing is used up, and
   wherein steps (a) to (d) and/or (e) to (h) are optionally repeated.

23 The method of item 22, wherein welding the selected length of tubing comprises welding a piece of fresh tubing having a length equal to the selected length of tubing to the third tubing.

24. The method of any one of items 21-23, wherein the selected length of a tubing comprises a multiple of a first length, the first length including a mushrooming length, a gripper width, a gap, and a seal width, and welding the selected length of tubing comprises slicing a piece of the third tubing having the first length.

25. The method of any one of items 22-24, wherein steps (b) and (f) comprise determining the selected length of tubing so that a length of the second tubing is equal to a length of the third tubing after completing step (f).

26. The method of any one of items 22-25, further comprising repeating steps (b) through (f) multiple cycles.

27. The method of any one of items 22-26, further comprising determining the selected length of tubing in steps (b) and (f) based on a number of a latest cycle and a desired length for a tubing coupled to a container for liquid transfer in the latest cycle.

28. A device, comprising:
a welding mount configured to receive a first tube fluidically coupled to a first container and a second tube fluidically coupled to a second container;
a welding element configured to fluidically couple the first tube with the second tube by welding;
a pump configured to transfer a liquid from the first container to the second container;
and
a controller comprising a memory circuit storing instructions and a processor circuit configured to execute the instructions, wherein, upon executing the instructions, the controller causes the welding element to fluidically couple the first tube and the second tube, optionally via a fresh piece of tubing of a selected length.

29. The device of item 28, further comprising an interlock valve configured to avoid back contamination between the first container and the second container.

30. The device of item 28 or item 29, further comprising a sealing head and cutter configured to seal and cut the flow path,

31. The device of any one of items 28-30, wherein the controller is configured to activate the welding element over the first tube and the second tube.

32. The device of any one of items 28-31, wherein the controller is configured to seal a flow path between the first container and the second container.

33. The device of any one of items 28-32, wherein the device is further configured for use in bioprocessing of cells.

34. The device of any one of items 28-33, further comprising a peristaltic pump between the first tube and the second tube to prevent backflow between the first container and the second container.

35. The device of any one of items 28-34, further comprising an interlocking pinch valve between the first tube and the second tube to prevent backflow between the first container and the second container.

1. A method for sterile automated liquid transfer, comprising:
(a) placing a first tube and a second tube into a welding mount, wherein the first tube is connected to a first container and the second tube is connected to a second container;
(b) welding the first tube and the second tube to form a first sterile fluidical connection between the first container and the second container;
(c) transferring a liquid between the first container and the second container via the first sterile fluidical connection;
(d) sealing and cutting the first fluidical connection between the first container and the second container to disconnect the first sterile fluidical connection;
wherein the steps (a)-(d) are operated automatically by a controller.

2. The method of claim 1, wherein the method further comprises:
(e) placing a third tube into the welding mount, wherein the third tube is connected to a third container;
(f) welding the first or second tube and the third tube to form a second sterile fluidical connection between the first container and the third container or between the second container and the third container;
(g) transferring a liquid between the first container and the third container or between the second container and the third container via the second sterile fluidical connection; and
(h) sealing the second fluidical connection between the connected first and third containers or between the connected second and third containers to disconnect the second sterile fluidical connection;
wherein steps (e)-(h) are operated automatically by a controller and
wherein steps (a)-(h) and/or (e)-(h) are optionally repeated.

3. The method of claim 1, wherein welding the first tube to the second tube comprises welding a fresh portion of a tube of a pre-selected length in-between the first tube and the second tube, thereby connecting the first tube and second tube.

4. The method of claim 1, wherein welding the first tube and the second tube comprises
(i) forming two separate sterile connections in the first tube and the second tube with a heated welder blade, a laser or a cold blade combined with a heating element, preferably a heated welder blade, and/or
(ii) welding, on one side of an existing weld, a selected length of a tubing, and adding a new weld to the opposite side of the existing weld until the selected length of tubing is used up.

5. The method of claim 4, wherein the initial length of the tubing prior to the welding is maintained.

6. The method of claim 2, wherein in step (b) and/or step (f), welding the first tube and the second tube, or welding the first or second tube and the third tube comprises forming two separate sterile connections in the first tube and the second tube with a spool piece, or forming two separate sterile connections in the first or second tube and the third tube with a spool piece with a welder single blade, and wherein the spool piece prevents backflow contamination between the first container and the second container, or between the first or second container and the third container.

7. The method of claim 5, wherein the initial length of the tube between the first container and the second container or the first container and the third container is substantially maintained.

8. The method of claim 2, wherein
(i) the controller comprises a memory circuit and a processor circuit, the memory circuit storing instructions which, when executed by the processor circuit, cause steps (a)-(d) and/or (e)-(h) to be performed automatically;
(ii) sealing a flow path between the first container and the second container, and/or between the first or second container and the third container comprises creating, for the first tube, two separate tubing seals, and the second tube with a single sealing head, and/or for the first or second tube, two separate tubing seals, and the third tube with a single sealing head;
(iii) step (a) comprises moving the first container or second container to a connection interface; and/or
(iv) the first container comprises a cell culture medium and the second and/or third container comprises cultured cells, and wherein the cell culture medium is transferred from the first container to the cultured cells.

9. The method of claim 1, wherein transferring a liquid between the first container and the second container comprises
(i) interlocking one or more pinch valves and/or a peristaltic pumps;
(ii) slightly rotating the peristaltic pump to create a positive or a negative pressure in one of the first tube or the second tube prior to releasing the one or more pinch valves to cause a positive or a negative pressure in one of the first tube or the second tube;
(iii) rotating a peristaltic pump between two interlocking valves prior to activating the interlocking valves; and/or
(iv) pumping the liquid from the first container to the second container or pumping the liquid from the first or second container to the third container.

10. The method of claim 1, wherein transferring a liquid between the first container and the second container comprises transferring a solution including a nucleic acid or a viral particle comprising such, to a cell culture for transducing a plurality of cells therein, optionally wherein the nucleic acid encodes a chimeric receptor.

11. The method of claim 8, wherein the cell culture comprises immune cells, which optionally
(i) are T cells; and/or
(ii) express the chimeric receptor.

12. The method of claim 2, wherein
(i) the first container is a cell culture vessel, the second and/or third container is a designation bag, and transferring a liquid between the first container and the second container comprises transferring a culture medium or a plurality of cells into the designation bag from a cell culture vessel;
(ii) the second container is a cell culture vessel, and wherein the method further comprises transferring liquid between the cell culture vessel and the third container; and/or
(iii) loading the first tube, the second tube, and optionally the third tube within the welding mount comprises robotically loading the first tube, the second tube, and the third tube within the welding mount.

13. A method for sterile automated connection and liquid transfer, comprising:
(a) placing a first tube and a second tube into a welding mount, wherein the first tube is connected to a first container and the second tube is connected to a second container;
(b) welding the first tube and the second tube to form a first sterile fluidical connection between the first container and the second container;
(c) transferring a liquid between the first container and the second container via the first sterile fluidical connection;
(d) sealing the first fluidical connection between the first container and the second container to disconnect the first sterile fluidical connection;
wherein steps (b) and (d) comprise welding, on one side of an existing weld, a spool piece of a selected length, and adding new welds to the added tubing thereby shortening the length of the spool piece,
preferably, wherein steps (b) and (d) comprise welding, on one side of an existing weld, a selected length of a tubing, and adding new welds to the added tubing until the selected length of tubing is used up;
optionally wherein the method further comprises:
(e) placing a third tube into the welding mount, wherein the third tube is connected to a third container;
(f) welding the first or second tube and the third tube to form a second sterile fluidical connection between the first container and the third container or between the second container and the third container;
(g) transferring a liquid between the first container and the third container or between the second container and the third container via the second sterile fluidical connection; and
(h) sealing the second fluidical connection between the connected first and third tubes or between the connected second and third tubes and cutting the seal to disconnect the second sterile fluidical connection,
wherein steps (e) and (f) comprise welding, on one side of an existing weld, a selected length of a tubing, and adding new welds to the added tubing until the selected length of tubing is used up, wherein steps (a) to (d) and/or (e) to (h) are optionally repeated.

14. The method of claim 13, wherein
(i) welding the selected length of tubing comprises welding a piece of fresh tubing having a length equal to the selected length of tubing to the third tubing;
(ii) the selected length of a tubing comprises a multiple of a first length, the first length including a mushrooming length, a gripper width, a gap, and a seal width, and welding the selected length of tubing comprises slicing a piece of the third tubing having the first length;
(iii) steps (b) and (f) comprise determining the selected length of tubing so that a length of the second tubing is equal to a length of the third tubing after completing step (f);
(iv) the method further comprises repeating steps (b) through (f) multiple cycles; and/or
(v) the method further comprises determining the selected length of tubing in steps (b) and (f) based on a number of a latest cycle and a desired length for a tubing coupled to a container for liquid transfer in the latest cycle.

15. The method of claim 13, wherein all of the steps (a)-(d) are operated automatically by a controller.

16. A device, comprising:
(i) a welding mount configured to receive a first tube fluidically coupled to a first container and a second tube fluidically coupled to a second container;
(ii) a welding element configured to fluidically couple the first tube with the second tube by welding;
(iii) a pump configured to transfer a liquid from the first container to the second container; and
(iv) a controller comprising a memory circuit storing instructions and a processor circuit configured to execute the instructions, wherein, upon executing the instructions, the controller causes the welding element to fluidically couple the first tube and the second tube, optionally via a spool piece of a selected length;
optionally wherein the device is further configured for use in bioprocessing of cells.

17. The device of claim 16, wherein the controller automatically operates the loading of the welding mount, welding, transfer of fluid, sealing and disconnection.

18. The device of claim 16, wherein the welding element comprises clamps and/or holders and cutting device, preferably a heated welder blade, a laser or a cold blade.

19. The device of claim 18, wherein the device comprises a sealing device, preferably, whereas the sealing device is combined with the cutting device, more preferably wherein the combined cutting and sealing device is a heated welder blade or a laser, whereas the sealing device and cutting device is configured to seal and cut the flow path.

20. The device of claim 16, further comprises:
(v) a welding mount configured to receive a spool piece of a selected length; and
(vi) a single or plurality of welding elements configured to fluidically couple the first tube with one side of the spool piece and the other side of the spool piece with the second tube by welding;
wherein all of (i)-(iii) and/or (v)-(vi) are automatically operated by (iv).

21. The device of claim 16, further comprising
(i) an interlock valve configured to avoid back contamination between the first container and the second container;
(ii) a peristaltic pump between the first tube and the second tube to prevent backflow between the first container and the second container; and/or
(iii) an interlocking pinch valve between the first tube and the second tube to prevent backflow between the first container and the second container.

22. The device of claim 16, wherein the controller is configured
(i) to automatically activate the welding element over the first tube and the second tube; and/or
(ii) to automatically seal a flow path between the first container and the second container.
